(19) United States
(12) Patent Application Publication
(10) Pub. No.: US 2024/0354667 A1
(43) Pub. Date: Oct. 24, 2024

(54) METHOD FOR EVALUATING NET ECOSYSTEM PRODUCTIVITY BASED ON PHOTOSYNTHETICALLY ACTIVE RADIATION ENERGY BALANCE

(71) Applicant: Institute of Atmospheric Physics, Chinese Academy of Scicences, Beijing (CN)

(72) Inventor: Jianhui Bai, Beijing (CN)

(21) Appl. No.: 18/538,003

(22) Filed: Dec. 13, 2023

(30) Foreign Application Priority Data
Apr. 24, 2023 (CN) 2023104457871

(51) Int. Cl. G06Q 10/063

(52) U.S. Cl. CPC G06Q 10/063

(57) ABSTRACT

A method for evaluating net ecosystem productivity (NEP) based on photosynthetically active radiation (PAR) energy balance is provided, and the method includes: determining a quantitative relationship among PAR, an ecosystem respiration term, a photochemical term, and a diffusion term based on a PAR energy balance principle at a horizontal plane above the canopy level; determining relative contributions of the PAR, the photochemical term, the diffusion term, and PAR reflection at the top of the atmosphere to the ecosystem respiration term respectively, based on the quantitative relationship, thereby constructing a 3-factor model of ecosystem respiration; and evaluating NEP by the 3-factor model of ecosystem respiration based on gross primary productivity (GPP). The method simplifies complex processes involved in the calculation of ecosystem respiration and NEP in plants, soil, and atmosphere, therefore saving computational resources and time.

### TECHNICAL FIELD

The disclosure relates to the technical field of evaluating net ecosystem productivity (NEP), in particular to a method for evaluating NEP based on photosynthetically active radiation (PAR) energy balance.

### BACKGROUND

NEP generally refers to a portion obtained by subtracting photosynthetic products consumed by heterotrophic respiration from net primary productivity (NPP), that is, a difference value between NPP of an ecosystem and heterotrophic respiration (soil and litter), which characterizes a rate of change in net carbon flux or carbon storage between land and atmosphere.

NEP is not only an important characteristic quantity of the ecosystem, but also a physical quantity of carbon exchange between a terrestrial ecosystem and the atmosphere. Without considering influences of various disturbances, a value of NEP indicates net carbon exchange of the terrestrial ecosystem (that is, quantities of carbon sources and sinks). NEP is limited by constraints including an atmospheric concentration of carbon dioxide, species composition, climate conditions, nutrients and other conditions, therefore, how to accurately obtain the NEP is of practical significance.

Calculation models of the NEP in the related art can be summarized into two aspects: 1) complex models, which need to consider detailed expression of various complex processes such as cycles and storages of energy, carbon, nitrogen, and water in plants and soils, and use many parameters, assumptions and corresponding parameters; 2) empirical models, which are proposed considering that calculation processes of the complex models are too simple to capture and describe main processes and related mechanisms of NEP reasonably and effectively. However, there are large uncertainties or biases in the calculation of NEP and ecosystem respiration for the two types of models. These models can only describe NEP in a single direction, and lack quantitative and accurate expression of interactions between multiple factors and processes. Therefore, a novel method for evaluating the NEP is urgently needed for meeting technical requirements of the NEP in the related art.

### SUMMARY

In order to solve technical problems in the related art, a purpose of the disclosure is to provide a method for evaluating NEP based on PAR energy balance. NEP is evaluated based on measurement data (solar radiation and meteorological parameters, ecosystem respiration (Re), NEP, and gross primary productivity (GPP)) of a research station and a PAR energy balance principle, which can meet the technical requirements of the NEP in the related art.

In order to achieve the above purpose, the disclosure provides a method for evaluating NEP based on PAR energy balance, and the method includes:
determining a quantitative relationship among the PAR, an ecosystem respiration term, a photochemical term, and a diffusion term based on a PAR energy balance principle of a horizontal plane above a canopy level;
determining relative contributions of the PAR, the photochemical term, the diffusion term, and an atmospheric top reflection term to the ecosystem respiration term based on the quantitative relationship, and constructing a 3-factor model of ecosystem respiration based on the relative contributions; and
evaluating, based on GPP, the NEP by using the 3-factor model of ecosystem respiration.

In an embodiment, the method for evaluating NEP based on PAR energy balance includes: before the determining the quantitative relationship, performing standardization processing on parameters for determining the quantitative relationship;
the parameters for determining the quantitative relationship include net ecosystem exchange (NEE), a solar elevation angle, and the PAR, and the standardization processing includes:
determining a measurement value of the NEE to be less than two times standard deviation;
determining a value of the solar elevation angle to be greater than 15 degrees; and
determining a measurement value of the PAR to be less than a PAR value at the top of the atmosphere.

In an embodiment, in the process of determining the quantitative relationship, the photochemical term is expressed as:

$${e}^{- k w m} = 1 - Δ {SI}_{0} cos Z$$

where I₀ represents a solar constant, Z represents a solar zenith angle, A S=0.172 (mW×0.1×60)^{0.303}, k represents a water vapor absorption coefficient, m represents an air mass, and W represents a water vapor content in an atmospheric column.

In an embodiment, in the process of determining the quantitative relationship, the diffusion term is expressed as e^{-S/Q}; where S represents a solar diffuse radiation and Q represents a global solar radiation.

In an embodiment, in the process of determining the quantitative relationship, the ecosystem respiration term is expressed as e^{-0.1bRetm}, where b represents an attenuation coefficient, Re represents the ecosystem respiration, and t represents sampling time.

In an embodiment, in the process of determining the quantitative relationship, the quantitative relationship is expressed as:

$$PAR = {A}_{1} {e}^{- 0.1 b R e t m} cos Z + {A}_{2} {e}^{- k w m} cos Z + {A}_{3} {e}^{- S / Q} + {A}_{0}$$

where A₁, A₂, and A₃ represent values of the ecosystem respiration item, the photochemical item, and diffusion item at the top of the atmosphere, respectively; and A₀ represents PAR reflection at the top of the atmosphere.

In an embodiment, in a process of constructing the 3-factor model of ecosystem respiration, the 3-factor model of ecosystem respiration is expressed as:

$${e}^{- 0.1 b R e t m} cos Z = {B}_{1} P A R + {B}_{2} {e}^{- k w m} cos Z + {B}_{3} {e}^{- S / Q} + {B}_{0}$$

where B₁, B₂, B₃, and B₀ represent the relative contributions of the PAR, the photochemical term, the diffusion term, and the PAR reflection at the top of the atmosphere to the ecosystem respiration term, respectively; B₁, B₂, B₃, and B₀ are a positive value, a positive value, a negative value, and a positive value, respectively.

In an embodiment, the evaluating, based on GPP, the NEP by using the 3-factor model of ecosystem respiration includes:
constructing, a first model for daytime evaluation and a second model for nighttime evaluation based on the 3-factor model of ecosystem respiration, wherein the second model is constructed based on the photochemical term;
acquiring daytime measurement data based on the first model, the second model, and the GPP, and evaluating daytime NEP based on the daytime measurement data, wherein the second model is configured to acquire daytime dark respiration based on the daytime measurement data; and
acquiring nighttime measurement data based on the second model and the GPP, and evaluating nighttime NEP.

In an embodiment, a system for evaluating NEP based on PAR energy balance includes:
a data collection module, configured to collect daytime measurement data and nighttime measurement data; and
an evaluation module, configured to evaluate NEP based on the daytime measurement data, the nighttime measurement data, a 3-factor model of ecosystem respiration, and GPP; wherein a quantitative relationship among PAR, an ecosystem respiration term, a photochemical term, and a diffusion term is determined based on a PAR energy balance principle of a horizontal plane above a canopy level; and relative contributions of the PAR, the photochemical term, the diffusion term, and an atmospheric top reflection term to the ecosystem respiration term is respectively determined based on the quantitative relationship, thereby constructing a 3-factor model of ecosystem respiration.

In an embodiment, the method for evaluating NEP based on PAR energy balance further includes: applying the NEP in policy and strategic guidance for ensuring the safety of ecological environment.

The disclosure has following technical effects.

Calculation results of the disclosure are reliable (a calculated standard deviation is less than a standard deviation of the measured value). Parameters of the empirical model used in the disclosure are easy to obtain from conventional stations.

The disclosure uses fewer parameters and hypotheses.

The disclosure simplifies complex processes involved in the calculation of ecosystem respiration and NEP in plants, soil, and atmosphere, therefore saving computational resources and time.

### BRIEF DESCRIPTION OF DRA WINGS

In order to provide a clearer explanation of embodiments of the disclosure or the technical solutions in the related art, a brief introduction will be given to drawings required in the embodiments. It is apparent that the drawings in the following description are only some embodiments of the disclosure. For ordinary those skill in the art, other drawings can be obtained based on these drawings without any creative work.

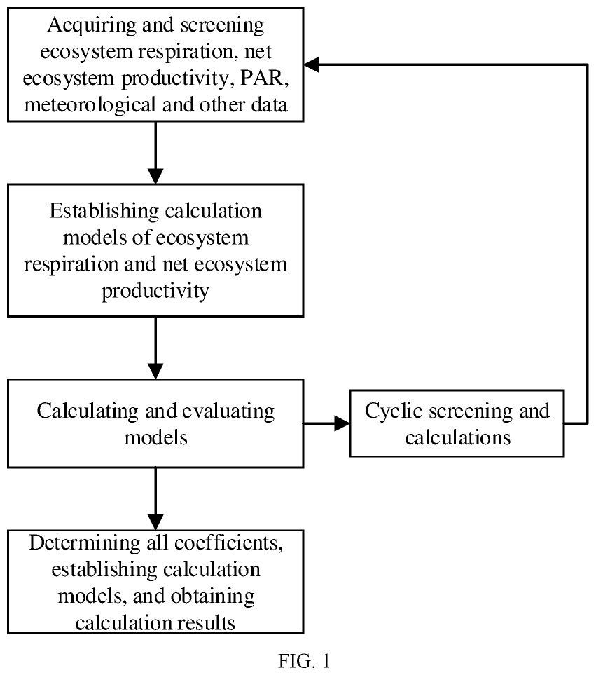

FIG. 1 illustrates a specific implementation flowchart of the disclosure.

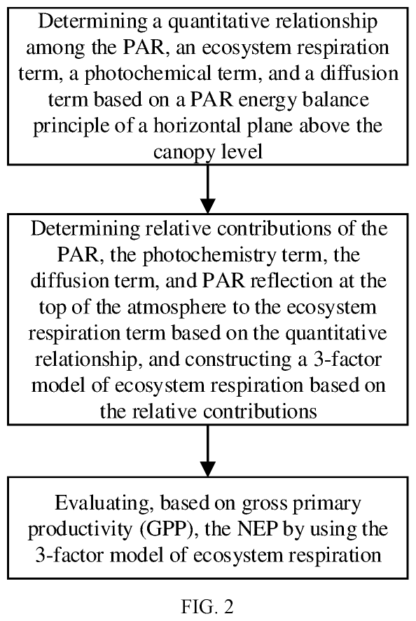

FIG. 2 illustrates a flowchart of the method of the disclosure.

### DETAILED DESCRIPTION OF EMBODIMENTS

In order to make the purpose, technical solutions, and advantages of embodiments of the disclosure clearer, the following will provide a clear and complete description of the technical solutions in the embodiments of the disclosure in conjunction with drawings. Apparently, the described embodiments are only a part of the embodiments of the disclosure, not all of them. Components of the embodiments of the disclosure, typically described and shown in the accompanying drawings, can be arranged and designed in various different configurations. Therefore, the detailed description of the embodiments of the disclosure provided in the drawings is not intended to limit the scope of the disclosure, but only to represent the selected embodiments of the disclosure. Based on the embodiments of the disclosure, all other embodiments obtained by those skilled in the art without creative work should fall within the scope of protection of the disclosure.

As shown in FIG. 1 and FIG. 2, in order to achieve the evaluation of NEP, the disclosure provides a calculation model for the NEP based on a PAR energy balance principle. The purpose of the calculation model is to provide reliable and accurate calculation data of the NEP and ecosystem respiration using fewer input parameters (four parameters which can be measured by experimental stations). The calculation data includes average and cumulative values at hourly, daily, monthly, annual, and multi-year scales (especially calculation results at the hourly is scale). With this calculation model, a standard deviation of calculated values is close to or less than a standard deviation of measured values, and a calculation error (such as root mean square error) is consistent with that of commonly used complex models. The calculation method of the disclosure can simplify complex calculations, does not need to use a large number of parameters used in commonly used models (complex models and experiential models), reduce the uncertainty (calculation error) of calculation results caused by the use of many assumed parameters (since many processes are unclear, the assumed parameters are used for calculation), and save calculation time and resources. Based on the PAR energy balance principle, the calculation model of the disclosure can better express and reveal main processes (PAR, atmosphere, plants, land, etc.) involved in NEP and ecosystem respiration, and the complex mechanisms of their interactions. The calculation model clearly reveals the relationship among control factors such as ecosystem respiration, NEP, PAR, water vapor, and diffuse radiation; some of mechanisms revealed by the calculation model are consistent with results of complex models commonly used internationally. The calculation method is simple and practical, easy to promote and apply to various ecosystems. By utilizing daily measurement data from conventional stations and previously developed GPP calculation models, it is possible to accurately estimate the primary productivity of ecosystems. Based on an acquisition situation (with or without direct or diffuse radiation) of experimental station data, it is possible to flexibly choose a 3-factor calculation model or a double-factor calculation model to obtain calculation results of NEP, ecosystem respiration, and others.

NEP involves numerous and complex processes such as plants, soil, water, nutrition, atmosphere, and their interactions. Many specific processes are currently unclear, and there are still many assumptions and parameterization schemes. The calculation method of the disclosure is based on the PAR energy balance principle to process the main processes mentioned above, thereby obtaining accurate calculation values of NEP.

The calculation method of NEP includes two parts: 1) the calculation model of ecosystem respiration; and 2) the calculation model of NEP.

Based on change laws and measurement data of parameters such as PAR, meteorology (temperature, humidity, and ground water vapor pressure), ecosystem respiration, NEP, and others, standards of setting values for ecosystem respiration, NEP, solar altitude angle (expressed as h in a formula), atmospheric substance content, atmospheric state (expressed as S/Q in a formula), and others are determined. The standards of setting values include: 1) for NEE, selecting data less than two standard deviations; 2) for solar elevation angles, selecting a value greater than 15 degrees; 3) for the PAR, a measurement value of the PAR being less than an atmospheric top value. The measurement value of the PAR is about 531.5 watts per square meter (W/m²) obtained from internationally recognized visible light radiation data. All other parameters are synchronized with the above data standards. To process the atmospheric state, the atmospheric state (expressed quantitatively as S/Q, where S and Q represent the solar diffuse radiation and the global solar radiation respectively) is divided into two types for processing, the two types include S/Q<0.5 and S/Q≥0.5.

After the above parameters are screened, the photochemical term, diffusion term, ecosystem respiration term, NEP term, and other items related to PAR transmission are calculated. The calculation model of ecosystem respiration is determined by using the PAR energy balance principle, in other words, each coefficient and constant in the calculation model of ecosystem respiration are determined.

Terms of the calculation model of ecosystem respiration are as follows.

The photochemical term e^{-kwm} is expressed as e^{-kwm}=1−ΔSI₀ cos Z, the solar constant I₀=1367 W/m², Z represents a solar zenith angle, ΔS=0.172 (mW×0.1×60)^{0.303} (unit is calories per square centimetre per minute (cal/cm²/min)), k represents a water vapor absorption coefficient (m⁻¹), m represents an air mass, and W represents a water vapor content in an atmospheric column (W=0.21E, E represents a ground water vapor pressure whose unit is hectopascal (hPa)).

The diffusion term is expressed as e^{-s/Q}, where S represents a solar diffuse radiation and Q represents a global solar radiation, the radiation unit is W/m², and e represents e-exponential.

The ecosystem respiration term is expressed as e^{-0.1bRetm}; where b represents an attenuation coefficient (b is set to 1), Re represents the ecosystem respiration (whose unit is mg CO₂/s), and t represents sampling time being 60 minutes.

### 1) The Calculation Model of Ecosystem Respiration

A quantitative relationship among PAR, an ecosystem respiration term, a photochemical term, and a diffusion term is determined based on a PAR energy balance principle of a horizontal plane above a canopy level. The quantitative relationship is expressed as:

$$PAR = {A}_{1} {e}^{- 0.1 b R e t m} cos Z + {A}_{2} {e}^{- k w m} cos Z + {A}_{3} {e}^{- S / Q} + {A}_{0} ; \qquad 1$$

in the formula 1, A₁, A₂, and A₃ represent values of the ecosystem respiration item, the photochemical item, and diffusion item at an atmospheric top, respectively; and A₀ represents PAR reflection at the top of the atmosphere.

The formula 1 is transformed to obtain the calculation model (i.e., the 3-factor model) of ecosystem respiration, and the 3-factor model is expressed as:

$${e}^{- {0.1}_{b R e t m}} cos Z = {B}_{1} P A R + {B}_{2} {e}^{- k w m} cos Z + {B}_{3} {e}^{- S / Q} + {B}_{0} ; \qquad 2$$

in the formula 2, B₁, B₂, B₃, and B₀ the relative contributions of the PAR, the photochemical term, the diffusion term, and the PAR reflection at the top of the atmosphere to the ecosystem respiration term, respectively; B₁, B₂, B₃, and B₀ are a positive value, a positive value, a negative value, and a positive value respectively.

For the double-factor model of ecosystem respiration, the diffusion term in the formula 1 and the formula 2 is removed; the coefficients B₁, B₂, B₃, and B₀ are adjusted to C₁, C₂, C₃=0, and C₀; the purpose is to make adaptive adjustments according to a situation where the diffusion term is not considered in practical applications.

The calculation model of ecosystem respiration (expressed as formula 2) is used to calculate ecosystem respiration and various errors in calculated and measured values (including average value, absolute deviation, relative deviation, root mean square, standard deviation, etc.).

When the above calculation deviations does not achieve the expected effect (such as relative deviation<15%), the previous data screening and subsequent corresponding calculations are repeated until a satisfactory calculation result is achieved: the relative deviation<15%, the minimum calculation deviation (absolute deviation, relative deviation, root mean square, standard deviation, etc.).

The calculation model of ecosystem respiration includes a daytime calculation model and a nighttime calculation model. The daytime calculation model is established by using daytime measurement data; and dark respiration at daytime can be obtained by applying daytime measurement data to the nighttime calculation model of ecosystem respiration. The nighttime calculation model is established by using nighttime measurement data; specifically PAR=0, the diffusion term=0, and the nighttime calculation model only uses the photochemical term.

In addition, for the 3-factor model, B₁, B₂, B₃, and B₀ are a positive value, a positive value, a negative value, and a positive value respectively. For the double-factor model, C₁, C₂, and C₀ are positive values.

The calculation model of ecosystem respiration includes the calculation of ecosystem respiration for two atmospheric states (S/Q<0.5 and S/Q≥0.5), different coefficients and constants are used for each atmospheric state to comprehensively describe and calculate ecosystem respiration under all weather conditions (including S/Q<0.5 and S/Q≥0.5). The uses of 3-factor and double-factor models of ecosystem respiration can obtain relatively consistent ecosystem respiration calculation results, including average and cumulative values. In addition, various calculation errors are also relatively close.

### 2) NEP Calculation Model

For the NEP calculation model, a previously developed GPP calculation model and an ecosystem respiration calculation model are used to calculate NEP, that is, NEP=GPP−ecosystem respiration. The NEP calculation model can accurately calculate the NEP, and the NEP calculation model can capture basic characteristics and change laws such as hourly, daily, monthly, and annual changes of NEP.

The NEP calculation model is established based on PAR energy utilization and is suitable for calculating NEP in various ecosystems, and its application range is relatively broad, but it needs to be determined based on measured data (i.e., coefficients related to each process) from experimental stations.

Embodiment 1: The methods mentioned above and the calculation model of ecosystem respiration (note: synchronous data used in the development of empirical models), the ecosystem respiration of subtropical coniferous forests in China is calculated under actual weather conditions (classified according to S/Q). The calculation results are as follows.

Regrading to the result of hourly average value calculated by the 3-factor model at daytime (S/Q<0.5, 2013-2014), the relative deviation between calculated and measured values is 24.31%, the normalized root mean square error (NMSE) is 0.085 mg CO₂/m²/s, the mean bias error (MBE) is 0.05 mg CO₂/m²/s and 24.04%, the root mean square error (RMSE) is 0.06 mg CO₂/m²/s and 29.37%. A ratio of the calculated value to the measured value is 1.001:1.

Regrading to the result of hourly average value calculated by the double-factor model at daytime (S/Q<0.5, 2013-2014), the relative deviation between calculated and measured values is 24.31%, the NMSE is 0.085 mg CO₂/m²/s, the MBE is 0.05 mg CO₂/m²/s and 24.04%, the RMSE is 0.06 mg CO₂/m²/s and 29.37%. A ratio of the calculated value to the measured value is 1.001:1.

Regarding to the result of hourly average value calculated by the 3-factor model at daytime (S/Q≥0.5, 2013-2014), the relative deviation between calculated and measured values is 68.45%, the NMSE is 0.582 mg CO₂/m²/s, the MBE is 0.12 mg CO₂/m²/s and 63.41%, the RMSE is 0.14 mg CO₂/m²/s and 76.72%. A ratio of the calculated value to the measured value is 1.009:1.

Regarding to the result of hourly average value calculated by the double-factor model at daytime (S/Q≥0.5, 2013-2014), the relative deviation between calculated and measured values is 72.46%, the NMSE is 0.638 mg CO₂/m²/s, the MBE is 0.12 mg CO₂/m²/s and 66.35%, the RMSE is 0.15 mg CO₂/m²/s and 80.30%. A ratio of the calculated value to the measured value is 1.010:1.

Regarding to the result of hourly average value at nighttime (2014), the relative deviation between calculated and measured values is 32.68%, the NMSE is 0.141 mg CO₂/m²/s, the MBE is 0.03 mg CO₂/m²/s and 19.81%, the RMSE is 0.05 mg CO₂/m²/s and 37.59%. A ratio of the calculated value to the measured value is 1.002:1.

Furthermore, when the calculation model of ecosystem respiration is applied to actual weather from 2013 to 2016, main calculation results are described below.

Regarding to the calculation result of the 3-factor model, a ratio of the annual total calculated value to the annual total measured value for the whole day (daytime+nighttime) is 1.40:1 (2013-2014) and 1.31:1 (2013-2016), which indicates that the annual total calculated values are overestimated by 40% and 31%, respectively.

Regarding to the calculation result of the double-factor model, a ratio of the annual total calculated value to the annual total measured value for the whole day (daytime+nighttime) is 1.36:1 (2013-2014) and 1.26:1 (2013-2016), which indicates that the annual total calculated values are overestimated by 36% and 26%, respectively.

When the NEP model is applied to actual weather (2013-2016), main calculation results including annual average calculation results of hourly values and annual total calculation results are described below.

Regarding to the annual average calculation result calculated by the 3-factor model, for the years 2013-2014, a ratio of the calculated value to the measured value is 0.93:1, standard deviations of the calculated and measured values are 0.245 mg CO₂/m²/s and 0.293 mg CO₂/m²/s respectively; for the years 2013-2016, a ratio of the calculated value to the measured value is 1.22:1, standard deviations of the calculated and measured values are 0.251 mg CO₂/m²/s and 0.299 mg CO₂/m²/s respectively.

Regarding to the annual average calculation result calculated by the double-factor model, for the years 2013-2014, a ratio of the calculated value to the measured value is 0.99:1, standard deviations of the calculated and measured values are 0.250 CO₂/m²/s and 0.293 CO₂/m²/s respectively; for the years 2013-2016, a ratio of the calculated value to the measured value is 1.27:1, standard deviations of the calculated and measured values are 0.254 CO₂/m²/s and 0.299 CO₂/m²/s respectively.

Regarding to the annual total calculation result calculated by the 3-factor model, for the years 2013-2014, a ratio of the calculated value to the measured value is 0.93:1; for the years 2013-2015, a ratio of the calculated value to the measured value is 1.07:1.

Regarding to the annual total calculation result calculated by the double-factor model, for the years 2013-2014, a ratio of the calculated value to the measured value is 0.99:1; for the years 2013-2015, a ratio of the calculated value to the measured value is 1.12:1.

In practical applications of the empirical model, the daily, monthly, and annual average calculation results, daily, monthly, and annual total calculation results are similar to the results of hourly values; and these will not be described in detail. In the empirical model, the standard deviations of calculated values (hourly, daily, monthly average, etc.) and measured values are relatively close.

Although embodiments of the disclosure have been shown and described above, it can be understood that the above embodiments are exemplary and cannot be understood as limitations to the disclosure. Those of ordinary skill in the art can make changes, modifications, substitutions, and variations to the above embodiments within the scope of the disclosure.

Those skilled in the art should understand that embodiments of the disclosure can be provided as methods, systems, or computer program products. Accordingly, the disclosure can be implemented in the form of an entirely hardware embodiment, an entirely software embodiment or an embodiment combining software and hardware. Furthermore, the disclosure may take the form of a computer program product embodied in one or more computer-usable storage media (including, but not limited to, magnetic disk storage, CD-ROM, optical storage, etc.) containing computer-usable program codes.

What is claimed is:

1. A method for evaluating net ecosystem productivity (NEP) based on photosynthetically active radiation (PAR) energy balance, comprising:
determining a quantitative relationship among the PAR, an ecosystem respiration term, a photochemical term, and a diffusion term based on a PAR energy balance principle of a horizontal plane above a canopy level;
determining relative contributions of the PAR, the photochemical term, the diffusion term, and an atmospheric top reflection term to the ecosystem respiration term based on the quantitative relationship, and constructing a 3-factor model of ecosystem respiration based on the relative contributions; and
evaluating, based on gross primary productivity (GPP), the NEP by using the 3-factor model of ecosystem respiration.

2. The method for evaluating NEP based on PAR energy balance as claimed in claim 1, wherein the method for evaluating NEP based on PAR energy balance comprises: before the determining the quantitative relationship, performing standardization processing on parameters for determining the quantitative relationship;
the parameters for determining the quantitative relationship comprise net ecosystem exchange (NEE), a solar elevation angle, and the PAR, and the standardization processing comprises:
determining a measurement value of the NEE to be less than two times standard deviation;
determining a value of the solar elevation angle to be greater than 15 degrees; and
determining a measurement value of the PAR to be less than an atmospheric top value.

3. The method for evaluating NEP based on PAR energy balance as claimed in claim 2, wherein in the process of determining the quantitative relationship, the photochemical term is expressed as:
$${e}^{- k w m} = 1 - Δ {SI}_{0} cos Z$$
where I₀ represents a solar constant, Z represents a solar zenith angle, Δ S=0.172 (mW×0.1×60)^{0.303}, k represents a water vapor absorption coefficient, m represents an air mass, and W represents a water vapor content in an atmospheric column.

4. The method for evaluating NEP based on PAR energy balance as claimed in claim 3, wherein in the process of determining the quantitative relationship, the diffusion term is expressed as e^{-S/Q}; where S represents a solar diffuse radiation and Q represents a global solar radiation.

5. The method for evaluating NEP based on PAR energy balance as claimed in claim 4, wherein in the process of determining the quantitative relationship, the ecosystem respiration term is expressed as e^{-0.1bRetm}, where b represents an attenuation coefficient, Re represents the ecosystem respiration, and t represents sampling time.

6. The method for evaluating NEP based on PAR energy balance as claimed in claim 5, wherein in the process of determining the quantitative relationship, the quantitative relationship is expressed as:
$$PAR = {A}_{1} {e}^{- 0.1 b R e t m} cos Z + {A}_{2} {e}^{- k w m} cos Z + {A}_{3} {e}^{- S / Q} + {A}_{0}$$
where A₁, A₂, and A₃ represent values of the ecosystem respiration item, the photochemical item, and diffusion item at the top of the atmosphere, respectively; and A₀ represents PAR reflection at the top of the atmosphere.

7. The method for evaluating NEP based on PAR energy balance as claimed in claim 6, wherein in a process of constructing the 3-factor model of ecosystem respiration, the 3-factor model of ecosystem respiration is expressed as:
$${e}^{- 0.1 b R e t m} cos Z = {B}_{1} P A R + {B}_{2} {e}^{- k w m} cos Z + {B}_{3} {e}^{- S / Q} + {B}_{0}$$
where B₁, B₂, B₃, and B₀ represent the relative contributions of the PAR, the photochemical term, the diffusion term, and the PAR reflection at the top of the atmosphere to the ecosystem respiration term, respectively; B₁, B₂, B₃, and B₀ are a positive value, a positive value, a negative value, and a positive value, respectively.

8. The method for evaluating NEP based on PAR energy balance as claimed in claim 7, wherein the evaluating, based on GPP, the NEP by using the 3-factor model of ecosystem respiration comprises:
constructing, a first model for daytime evaluation and a second model for nighttime evaluation based on the 3-factor model of ecosystem respiration, wherein the second model is constructed based on the photochemical term;
acquiring daytime measurement data, based on the first model, the second model, and the GPP, and evaluating daytime NEP using the daytime measurement data, wherein the second model is configured to acquire daytime dark respiration using the daytime measurement data; and
acquiring nighttime measurement data, based on the second model and the GPP, and evaluating nighttime NEP.

9. The method for evaluating NEP based on PAR energy balance as claimed in claim 1, further comprising:
applying the NEP in policy and strategic guidance for ensuring the safety of ecological environment.
